(19) United States
(12) Patent Application Publication
(10) Pub. No.: US 2026/0076314 A1
(43) Pub. Date: Mar. 19, 2026

(54) Pollination Method

(71) Applicant: SUN'AGRI, PARIS (FR)

(72) Inventors: Gerardo Lopez Velasco, MONTPELLIER (FR); Damien Fumey, MONTPELLIER (FR)

(21) Appl. No.: 19/328,147

(22) Filed: Sep. 13, 2025

(30) Foreign Application Priority Data
Sep. 17, 2024 (FR) 2409882

(51) Int. Cl. A01G 17/00; A01G 7/00; G06V 20/10; H02S 20/30

(52) U.S. Cl. CPC A01G 17/005; A01G 7/00; H02S 20/30; G06V 20/188

(57) ABSTRACT

A method for pollinating plants (A), notably fruit trees, located underneath orientable photovoltaic collectors (C) is provided. The shade cast on the plants is modified by changing the orientation of the collectors. The orientation of the collectors is automatically acted on depending on the state of flowering such that the sunlight and/or exposure to the wind is increased in relation to a collectors control reference applied when the flowering is not mature or has not commenced, to encourage the activity of pollinators and/or promote the effect of the wind on the pollination.

### RELATED APPLICATION

This application claims the benefit of priority from French Patent Application No. 24 2409882, filed on Sep. 17, 2024, the entirety of which is incorporated by reference.

### TECHNICAL FIELD

The present invention relates to pollination methods.

### PRIOR ART

Pollination is an essential fruit-production mechanism for the vast majority of flowering plants on the planet. This process consists in transferring pollen from the male reproductive system to the female part of the flower. Pollen vectors, such as the wind and some insects, are therefore necessary to go about this operation.

Application CN113424741 relates to a method for cultivating kiwi fruits under a photovoltaic power plant comprising non-orientable photovoltaic panels. The latter protect the plantings from the sun in summer and prevent rainwater from penetrating the trunks, thereby being able to effectively prevent the propagation of canker. During pollination, the south-facing side of the same plant is naturally pollinated, whereas on the north-facing side artificial pollination must be implemented.

Application CN116784130 discloses a greenhouse which incorporates, among other things, the generation of photovoltaic energy. The greenhouse comprises a movable-rail pollination system.

### SUMMARY OF THE INVENTION

There is a need to further improve methods for pollinating plants, notably fruit trees, that are located underneath photovoltaic collectors, in order to optimize the pollination process while still generating electrical energy.

The invention aims to meet this objective and, according to one of its aspects, relates to a method for pollinating plants, notably fruit trees, located underneath orientable photovoltaic collectors, the shade cast on the plants being modified by changing the orientation of the collectors, in which method the orientation of the collectors is automatically acted on depending on the state of flowering such that the sunlight and/or exposure to the wind is increased in relation to a collectors control reference applied when the flowering is not mature or has not commenced, to encourage the activity of pollinators and/or promote the effect of the wind on the pollination.

“Mature flowering” is understood to mean that more than 20% of the flowers among a population of flowers of the plant in an area observable by a camera or a human operator can be pollinated, a flower that can be pollinated being in a state of total or partial blossoming depending on the nature of the flower.

“Flowering that has not commenced” is understood to mean the absence of flowers that can be pollinated, or less than 20% of the flowers can be pollinated, among a population of flowers of the plant in an area observable by a camera or a human operator.

The invention thus makes it possible to benefit from the orientable nature of the photovoltaic collectors to encourage the activity of pollinators and/or promote the effect of the wind on the pollination when the flowering is mature.

With preference, the orientation of the photovoltaic collectors is controlled on the basis at least of data indicative of the phenological stages of the plants, notably the state of flowering, the indicators of the state of flowering including in particular the density of the flowers and/or their size and/or their colors and/or the size of the organs constituting them and/or the number of flowers that have bloomed in relation to the total number of flowers.

The phenological stage of a plant refers to the specific phase of its growth and development cycle. It describes where the plant is in its biological cycle depending on the weather conditions, the season and other environmental factors.

The phenological stages of a plant include multiple key steps such as dormancy, germination, plant development and flowering.

With preference, the orientation of the collectors is acted on while at the same time seeking to maximize the generation of electrical energy in relation to a reference not combined with the plant.

The collectors are advantageously controlled as a function of the outside temperature and/or on the day of the week, notably with a view to reducing the shade generated by the collectors at times when the demand for electrical energy is low.

In one embodiment, the photovoltaic collectors are oriented so as to generate the least shade possible on the plants to be pollinated during the period of mature flowering, the average light energy received by the plants and absorbed in the course of a day being notably at its highest during the periods of mature flowering.

At least one camera can be used to acquire images of the plants and track the state of flowering. The method notably comprises automatically processing the images from the camera to detect the state of flowering and to generate an information item on the basis of which the collectors are controlled.

The images taken by the camera are preferably compared to reference images indicating the stage of flowering.

The images taken by the camera can be segmented to identify the flowers. The images taken by the camera can be processed by colorimetry to analyze the colors of the flowers. The dimension and/or the color of the organs of the flower can be used as indicator to determine the stage of flowering.

With preference, if the images taken by the camera show that at least more than 20% of the flowers are ready to be pollinated, then the flowering is considered to be mature and a mode for controlling the collectors that promotes pollination is activated.

With preference, the state of flowering is periodically reevaluated during the period of flowering, notably every day.

In one embodiment, an application for a mobile terminal is made available to at least one user to allow at least one information item relating to the state of flowering to be entered, this information item being transmitted to a system for controlling the collectors.

With preference, the application is configured to automatically geolocate the mobile terminal, and the terminal transmits at least one information item relating to the state of flowering and/or to the activity of pollinators close to the terminal, and an information item about the location of the terminal.

At least one photograph of the plants can be taken with the mobile terminal, and this photograph can be automatically analyzed to deduce at least one information item therefrom regarding the state of flowering and/or the activity of pollinators, the photograph preferably being geolocated. In one embodiment, the presence of wind in the surroundings of the plants, notably the wind speed and direction, is detected by a wind sensor, notably an anemometer.

During the period of mature flowering and if the wind exceeds a predefined speed, the photovoltaic collectors are preferably oriented parallel to the slope of the ground.

The plants can be selected from the following: fruit trees, notably pome and stone fruit trees, in particular apple trees, pear trees, plum trees, apricot trees, fig trees, kiwi fruit trees, cherry trees and peach trees.

The invention also relates, according to another of its aspects, to a method for cultivating plants underneath orientable photovoltaic collectors, wherein these plants are cultivated while at the same time acting on the activity of pollinators of the plants by implementing the pollination method according to the invention.

### BRIEF DESCRIPTION OF THE DRAWING

The invention may be understood better from reading the following detailed description of nonlimiting implementation examples thereof, and from examining the appended drawing, in which:

FIG. 1 schematically shows a system for controlling the orientation of a photovoltaic collector according to the invention;

FIG. 2 is a schematic view of an electrical energy generation system comprising photovoltaic collectors for implementing the pollination method according to the invention; and

FIG. 3 is similar to FIG. 2, taking into account the wind as pollination contributor.

### DETAILED DESCRIPTION

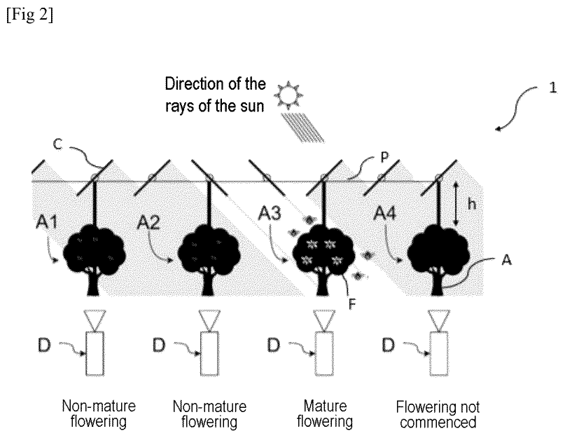

FIG. 2 schematically illustrates an electrical energy generation system 1 comprising a support structure P and orientable photovoltaic collectors C kept at a non-zero distance from the ground by the support structure P, and at a height h of fruit trees A, for example apple trees, which have flowers F and are located underneath the collectors C. The sun shade cast on the plants is modified by changing the orientation of the collectors C.

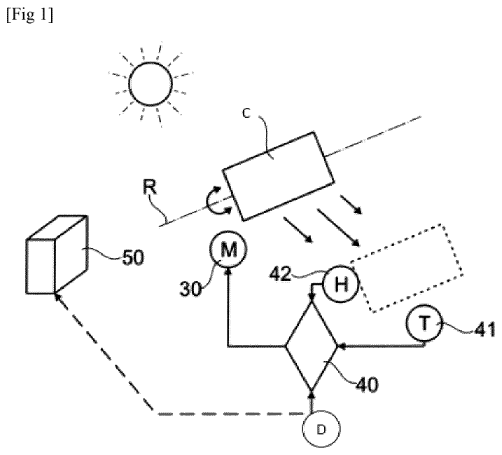

FIG. 1 shows one of these photovoltaic collectors C which is movable about an axis of rotation R. The photovoltaic sensor C is driven about the axis R by means of at least one actuator 30. For example, an actuator 30 is provided individually for each photovoltaic collector C. In a variant, one and the same actuator 30 can rotate a plurality of photovoltaic collectors C.

The actuators 30 each comprise for example one or more electric motors, and are made up for example of servo-actuators.

The position to be given to the photovoltaic collector C can be determined by a local computer 40 which is connected via any suitable power interface to the actuator 30.

The computer 40 preferably receives weather information, notably from one or more local sensors, for example a temperature sensor 41 and a humidity sensor 42. Other sensors can be added to examine the weather conditions, such as a rain gauge, anemometer, and/or a camera D for acquiring images and viewing the state of development of the plant and the state of flowering, and also one or more biosensors, where appropriate. Said camera D can be either supported by the structure P or by another separate structure, or by a drone.

The computer 40 can also exchange data, for example via a wireless phone network, with a remote server 50, which can for example provide the computer 40 with information about the coming weather. The local data relating to temperature and wind can thus come from a remote weather server.

The computer 40 can be implemented on the basis of any micro-computer or computer equipment allowing the orientation of the photovoltaic collectors C to be controlled as a function of one or more control laws that provide the orientation to be imposed on the photovoltaic collectors on the basis of data indicative of the phenological stages of the plants, notably the state of flowering, the indicators of the flowering state including in particular the density of the flowers F and/or their size and/or their colors and/or the size of the organs constituting them and/or the number of flowers that have bloomed in relation to the total number of flowers. The orientation to be imposed on the photovoltaic collectors C can also be controlled depending on the plant variety, the weather conditions, the activity of pollinators and/or the state of flowering. The computer 40 can be designed to process images sent by the camera D in order to determine the state of flowering.

As an alternative, a human operator observes the state of flowering, for example at an observation area, and if the degree of flowering corresponds to the existence of the desired population of flowers that can be pollinated, indicates the maturity of the flowering by means of a mobile application connected to the computer 40.

The computer 40 may comprise a calculation unit and a local memory in which local data relating to the plants and/or to their environment can be stored.

The memory of the computer can also contain automatic control parameters which regulate the orientation of the photovoltaic sensors C as a function of pollination targets. These parameters can change over time and as a function for example of the season, and prioritize pollinating or not pollinating the plants.

The control law(s) can be initially programmed into the computer 40, or alternatively be downloaded by the computer 40 from the remote server 50, or else be updated periodically by the remote server 50.

In one exemplary embodiment, the computer 40 operates autonomously. Depending on the season, on the sowing date, on the number of flowers present and/or on the state of flowering, and possibly on other parameters provided by the grower, it automatically controls the orientation of the photovoltaic collectors C daily or with another frequency such that the pollination target is met over a given period.

The computer 40 is designed to process the images acquired by the camera D and identify the state of flowering by comparing these images with reference images. The images acquired can be segmented to identify the flowers. The images taken by the camera can be processed by colorimetry to analyze the colors of the flowers. The dimension and/or the color of the organs of the flower can be used as indicator to determine the stage of flowering.

Depending on the state of flowering, the collectors C are oriented either to cast as much shade as possible on the trees A, or to minimize the shade cast by allowing light to pass and thus promote the activity of the pollinators, in the present case insects.

For example, if the images taken by the camera D show that the required population of flowers is ready to be pollinated, then the flowering is considered to be mature and a mode for controlling the collectors that promotes pollination is activated.

When this control mode is activated, the photovoltaic collectors C are for example oriented for several days to promote the passage of light and the effect of pollinators such as insects, notably bees, to the greatest possible extent. Then, once flowering has finished, the photovoltaic sensors C are for example controlled by activating the actuators 30 to take an orientation aiming to allow as little light as possible to pass, maximizing electricity generation.

FIG. 2 shows that, when the flowering is mature, the collectors C are oriented in the direction of the rays of the sun, thus casting as little shade as possible on the tree A3. By contrast, when the flowering is not mature or has not commenced yet, the collectors C are oriented such that as much shade as possible is cast on the trees A1, A2 and A4.

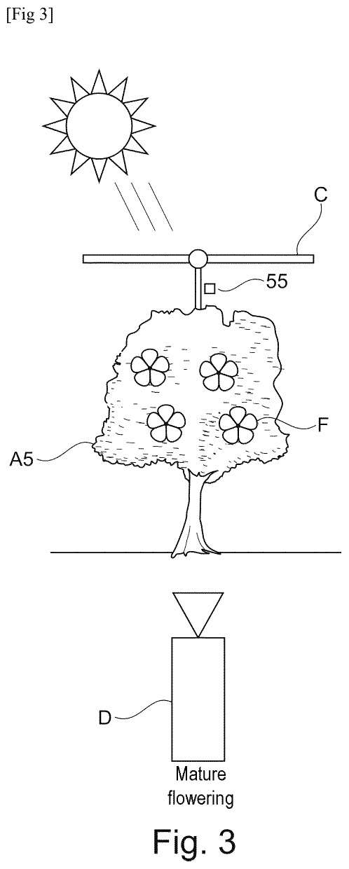

FIG. 3 shows a tree A5 in the state of mature flowering. The installation receives an information item about the speed of the wind from a weather server and/or comprises an anemometer 55 which supplies this information.

When the wind speed is suitable for pollination, the collector C is oriented horizontally so as to allow as much wind as possible to pass underneath the panels to promote the pollination of the flowers.

1. A method for pollinating plants located underneath orientable photovoltaic collectors, the shade cast on the plants being modified by changing an orientation of the collectors, said method comprising the steps of:
automatically acting on the orientation of the collectors depending on the state of flowering such that the sunlight and/or exposure to the wind is increased in relation to a collectors control reference applied when the flowering is not mature or has not commenced, to encourage the activity of pollinators and/or promote the effect of the wind on the pollination.

2. The method as claimed in claim 1, wherein the orientation of the photovoltaic collectors being controlled on the basis at least of data indicative of the phenological stages of the plants including the state of flowering, the indicators of the state of flowering including in particular the density of the flowers (F) and/or their size and/or their colors and/or the size of the organs constituting them and/or the number of flowers that have bloomed in relation to the total number of flowers.

3. The method as claimed in claim 1, wherein the orientation of the collectors is acted on while at the same time seeking to maximize the generation of electrical energy in relation to a reference not combined with the plants.

4. The method as claimed in claim 1, wherein the collectors are controlled as a function of the outside temperature and/or on the day of the week for reducing the shade generated by the collectors at times when the demand for electrical energy is low.

5. The method as claimed in claim 1, the photovoltaic collectors being oriented so as to generate the least shade possible on the plants to be pollinated during the period of mature flowering, the average light energy received by the plants and absorbed in the course of a day being notably at its highest during the periods of mature flowering.

6. The method as claimed in claim 1, at least one camera being used to acquire images of the plants and track the state of flowering, the method comprising automatically processing the images from the camera to detect the stage of flowering and to generate information on the basis of which the collectors are controlled.

7. The method as claimed in claim 6, the images taken by the camera being compared to reference images indicating the stage of flowering.

8. The method as claimed in claim 6, the images taken by the camera being segmented to identify the flowers.

9. The method as claimed in claim 6, the images taken by the camera being processed by colorimetry to analyze the colors of the flowers.

10. The method as claimed in claim 6, wherein, if the images taken by the camera show that more than 20% of the flowers are ready to be pollinated, then the flowering is considered to be mature and a mode for controlling the collectors that promotes pollination is activated.

11. The method as claimed in claim 1, wherein the state of flowering is periodically reevaluated during the period of flowering, notably every day.

12. The method as claimed in claim 1, wherein an application for a mobile terminal is made available to at least one user to allow at least one information item relating to the state of flowering to be entered, this information item being transmitted to a system for controlling the collectors.

13. The method as claimed in claim 12, wherein the application is configured to automatically geolocate the mobile terminal, and wherein the terminal transmits at least one information item relating to the state of flowering and/or to the activity of pollinators close to the terminal, and an information item about the location of the terminal.

14. The method as claimed in claim 12, wherein at least one photograph of the plants is taken with the mobile terminal, and wherein this photograph is automatically analyzed to deduce at least one information item therefrom regarding the state of flowering and/or the activity of pollinators, the photograph preferably being geolocated.

15. The method as claimed in claim 1, wherein the presence of wind in the surroundings of the plants, including the wind speed and direction, is detected by a wind sensor, including an anemometer.

16. The method as claimed in claim 15, wherein, during the period of mature flowering and if the wind exceeds a predefined speed, the photovoltaic collectors are oriented parallel to the slope of the ground.

17. The method as claimed in claim 1, the plants being selected from the following: fruit trees selected from the group consisting of: pome and stone fruit trees, apple trees, pear trees, plum trees, apricot trees, fig trees, kiwi fruit trees, cherry trees, and peach trees.

18. A method for cultivating plants underneath orientable photovoltaic collectors, wherein these plants are cultivated while at the same time acting on the activity of pollinators of the plants by implementing the pollination method as claimed in claim 1.
